(19) United States
(12) Patent Application Publication
(10) Pub. No.: US 2024/0307296 A1
(43) Pub. Date: Sep. 19, 2024

(54) SUBCUTANEOUS BIODEGRADABLE NALTREXONE IMPLANT AND ACCOMPANYING BEHAVIORAL PROGRAM FOR WEIGHT LOSS IN A PATIENT

(71) Applicant: Biocorrx, Anaheim, CA (US)

(72) Inventors: Lourdes Felix, Rancho Santa Margarita, CA (US); Brady James Granier, Valley Glen, CA (US)

(73) Assignee: Biocorrx, Anaheim, CA (US)

(21) Appl. No.: 18/426,299

(22) Filed: Jan. 29, 2024

Related U.S. Application Data
(63) Continuation of application No. 16/915,688, filed on Jun. 29, 2020, now abandoned.
Application No. 16/915,688 is a continuation of application No. 16/150,154, filed on Oct. 2, 2018, now abandoned.
(60) Provisional application No. 62/566,994, filed on Oct. 2, 2017.

(51) Int. Cl. A61K 9/00; A61K 31/485; A61P 3/04; G09B 19/00

(52) U.S. Cl. CPC A61K 9/0024; A61K 31/485; A61P 3/04; G09B 19/0092

(57) ABSTRACT

Embodiments of the present disclosure provide methods, systems, and apparatuses for aiding weight loss in a patient. According embodiments, an exemplary system for aiding weight loss in a patent comprises a subcutaneous biodegradable medical implant placed or injected in the patient, the biodegradable medical implant comprising naltrexone (C₂₀H₂₃NO₄) and capable of releasing the naltrexone from the subcutaneous biodegradable medical implant following the placement of the subcutaneous biodegradable medical implant in the patient; and an optional program comprising behavioral counseling and/or therapy delivered by a licensed professional to the patient.

### CROSS-REFERENCE TO RELATED APPLICATIONS

The present application is a continuation-in-part of U.S. application Ser. No. 16/915,688 filed on Jun. 29, 2020, now abandoned, which is a continuation of U.S. application Ser. No. 16/150,154, titled “SUBCUTANEOUS BIODEGRADABLE NALTREXONE IMPLANT AND ACCOMPANYING BEHAVIORAL PROGRAM FOR WEIGHT LOSS IN A PATIENT,” filed Oct. 2, 2018, now abandoned, which claims priority to U.S. Provisional Application Ser. No. 62/566,994, titled “SUBCUTANEOUS BIODEGRADABLE NALTREXONE IMPLANT AND ACCOMPANYING BEHAVIORAL PROGRAM FOR WEIGHT LOSS IN A PATIENT,” filed Oct. 2, 2017, the contents of which are incorporated herein by reference in their entirety.

### BACKGROUND

For decades, a significant portion of the earth's population have struggled with weight loss and obesity. Obesity and weight gain are the result of too much body fat in a patient and can lead to serious health conditions, including heart disease, stroke, metabolic syndrome, and diabetes. Obesity and weight gain are chronic conditions that can last several years or even be lifelong. Over the years countless weight loss programs and devices have been introduced with varying levels of success, and each having its own set of drawbacks. Through applied effort, ingenuity, and innovation, many of these identified problems have been solved by developing solutions that are included in embodiments of the present disclosure, many examples of which are described in detail herein.

### BRIEF SUMMARY

This specification relates to a subcutaneous biodegradable medical implant comprising naltrexone that, when implanted in a patient and optionally combined with behavioral counseling or therapy, aid in weight loss in the patient. The specification also relates to a subcutaneous non-biodegradable medical implant comprising naltrexone that, when implanted in a patient and optionally combined with behavioral counseling or therapy, aids in weight loss in the patient. The specification also relates to a subcutaneous semi-biodegradable medical implant comprising naltrexone that, when implanted in a patient and optionally combined with behavioral counseling or therapy, aids in weight loss in the patient.

In one embodiment, system for aiding weight loss in a patient comprises a subcutaneous biodegradable medical implant placed in the patient, the subcutaneous biodegradable medical implant comprising naltrexone. The implant is capable of releasing the naltrexone from the subcutaneous biodegradable medical pellet(s) following the placement of the subcutaneous biodegradable medical implant in the patient. The system further comprises a program comprising behavioral program comprising one or more of behavioral counseling, nutritional counseling, and therapy delivered by one or more certified or licensed professionals to the patient. In some embodiments, the subcutaneous biodegradable, semi-biodegradable, and non-biodegradable medical implants comprise one or more pellets formed of naltrexone. In some embodiments, the present implants are in the form of rods.

It will be appreciated that the biodegradable embodiments of the present disclosure eliminate a need for removal of the implants from a patient.

In some embodiments, the implant releases dosage amount(s) of naltrexone into a bloodstream of the patient. In embodiments, the dosage amount(s) of naltrexone can be in an amount within the range of 200 mg to 4 grams. In some embodiments, the dosage amount(s) of naltrexone is in an amount of 1 gram or 1.1 grams. In some embodiments, the dosage amount(s) of naltrexone is in an amount of 2.2 grams.

In some embodiments, the dosage amount(s) of naltrexone can be in an amount within the range of 250 mg to 4 grams, 300 mg to 4 grams, 350 mg to 4 grams, 400 mg to 4 grams, 450 mg to 4 grams, 500 mg to 4 grams, 550 mg to 4 grams, 600 mg to 4 grams, 650 mg to 4 grams, 700 mg to 4 grams, 750 mg to 4 grams, 800 mg to 4 grams, 850 mg to 4 grams, 900 mg to 4 grams, 950 mg to 4 grams, 1 gram to 4 grams, 1.1 grams to 4 grams, 1.5 grams to 4 grams, 2 grams to 4 grams, 2.2 grams to 4 grams, 2.2 grams to 3 grams, 2 grams to 3 grams, 1.1 grams to 3 grams, 1 gram to 3 grams, 950 mg to 3 grams, 900 mg to 3 grams, 850 mg to 3 grams, 800 mg to 3 grams, 750 mg to 3 grams, 700 mg to 3 grams, 650 mg to 3 grams, 600 mg to 3 grams, 550 mg to 3 grams, 500 mg to 3 grams, 450 mg to 3 grams, 400 mg to 3 grams, 350 mg to 3 grams, 300 mg to 3 grams, 250 mg to 3 grams, 200 mg to 3 grams, 200 mg to 2 grams, 250 mg to 2 grams, 300 mg to 2 grams, 350 mg to 2 grams, 400 mg to 2 grams, 450 mg to 2 grams, 500 mg to 2 grams, 550 mg to 2 grams, 600 mg to 2 grams, 650 mg to 2 grams, 700 mg to 2 grams, 750 mg to 2 grams, 800 mg to 2 grams, 850 mg to 2 grams, 900 mg to 2 grams, 950 mg to 2 grams, 1 gram to 2 grams, 1.1 grams to 2 grams, 1.5 grams to 2 grams.

Non-limiting examples of dosage amount(s) of naltrexone in the presently disclosed implant include any dosage or amount in increments and/or combinations of 50 mg, 100 mg, 200 mg, 500 mg, 1 gram or 1.1 grams, and the like. It will be appreciated that dosages or amounts incrementally between those described above are within the scope of the present disclosure.

In some embodiments, the subcutaneous biodegradable medical implant comprises a single implant unit (or otherwise referred to as a pellet). For example, for a subcutaneous biodegradable medical implant configured to release a dosage amount of 400 mg of naltrexone into a patient's bloodstream, a single 400 mg biodegradable naltrexone pellet may be used.

In some embodiments, the subcutaneous biodegradable medical implant comprises two or more implant units (or otherwise referred to as pellets). For example, for a subcutaneous biodegradable medical implant configured to release a dosage amount of 400 mg of naltrexone into a patient's bloodstream, two (2) 200 mg biodegradable naltrexone pellets may be used.

In embodiments, the subcutaneous biodegradable medical implant biodegrades in the patient. In some embodiments, the subcutaneous biodegradable medical implant biodegrades after a period of about 30 days in the patient. In embodiments, the subcutaneous biodegradable medical implant biodegrades over a period of about several months in the patient. It will be appreciated that the time it takes to for an implant to biodegrade in a patient is dependent upon multiple factors including dosage, patient metabolism, external activity, and the like.

In some embodiments, the subcutaneous biodegradable medical implant is placed below a skin surface of the patient. In some embodiments, the subcutaneous biodegradable medical implant is below a skin surface of the patient and above a muscle fascia of the patient.

In some embodiments, the subcutaneous biodegradable medical implant is placed below a skin surface of a lower abdomen of the patient. In some embodiments, the subcutaneous biodegradable medical implant is placed below a skin surface of one or more of a hip, a leg, a back, and an arm of the patient. As mentioned above, in some embodiments the implants disclosed herein are placed below a skin surface of a patient and above a muscle fascia of the patient.

In some embodiments, delivering the program comprising behavioral counseling and/or therapy to the patient occurs prior to or from a placement time in the patient until a biodegradation time.

In some embodiments, delivering the program comprising behavioral counseling and/or therapy to the patient occurs prior to or from a placement time in the patient until after a biodegradation time.

In some embodiments, the program comprising behavioral counseling and/or therapy comprises nutritional therapy.

In some embodiments, a second subcutaneous biodegradable medical implant is placed into a patient subsequent to a biodegradation time of a first subcutaneous biodegradable medical implant.

In embodiments, the patient experiences a reduction in body mass index (BMI) of one of at least 2% after four weeks from a placement time in the patient, at least 3% after 8 weeks from a placement time in the patient, or at least 4.5% after 12 weeks from a placement time in the patient.

In embodiments, the patient experiences a reduction in body mass index (BMI) in a range from 1.7% to 2.7% after four weeks from a placement time in the patient.

In embodiments, the patient experiences a reduction in body mass index (BMI) in a range from 3% to 4.9% after eight weeks from a placement time in the patent.

In embodiments, the patient experiences a reduction in body mass index (BMI) in a range from 4.7% to 7.6% after twelve weeks from a placement time in the patient.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

Having thus described the disclosure in general terms, reference will now be made to the accompanying drawings, which are not necessarily drawn to scale, and wherein:

FIG. 1 is a diagram of a system for aiding weight loss in a patient according to embodiments of the present disclosure;

FIG. 2 is a diagram of an exemplary subcutaneous implant placed in a patient according to embodiments of the present disclosure;

FIG. 3A illustrates an exemplary subcutaneous implant for use with embodiments of the present disclosure;

FIG. 3B is a photograph of an exemplary subcutaneous implant for use with embodiments of the present disclosure;

FIG. 4A illustrates an exemplary process for placing a subcutaneous implant in a patient according to embodiments of the present disclosure;

FIG. 4B illustrates an exemplary process for placing a subcutaneous implant in a patient according to embodiments of the present disclosure;

FIG. 4C illustrates an exemplary process for placing a subcutaneous implant in a patient according to embodiments of the present disclosure;

FIG. 4D illustrates an exemplary process for placing a subcutaneous implant in a patient according to embodiments of the present disclosure;

FIG. 5 illustrates projected weight loss for the subjects based on collected data after 4 weeks of a patient study; and

FIG. 6 illustrates data representing side effects reported by the subjects of the patient study.

### DETAILED DESCRIPTION OF VARIOUS EMBODIMENTS

Various embodiments of the present disclosure now will be described more fully hereinafter with reference to the accompanying drawings, in which some, but not all embodiments of the disclosure are shown. Indeed, the disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

### Overview

Various embodiments of the disclosure generally relate to a subcutaneous biodegradable medical implant comprising naltrexone (i.e., naltrexone hydrochloride, naltrexone base) that, when implanted in a patient and combined with behavioral counseling, aids in weight loss in the patient.

### A. Naltrexone

Naltrexone is a prescription drug belonging to a class of drugs called opioid antagonists. It's commonly used to treat opioid use disorder and alcohol use disorder and to block the effects of opioids at the cellular level. It functions by blocking some of the effects of both opioids and alcohol at a brain cell receptor level. Naltrexone is known to minimize or block in a patient the euphoric effects of a drug such as heroine. Until recently, naltrexone was not thought of as effective in aiding weight loss in a patient.

The molecular formula for naltrexone is C201-123N04. It will be appreciated that naltrexone may also be referred to as Vivitrex, ReVia, N-Cyclopropylmethylnoroxymorphone, Vivitrol, Celupan, Naltrexonum, Naltrexona, Naltrel, N-Cyclopropylmethy 1-14-hydroxidihydromorphinone, among others. The present application applies to the use of the identified molecular formula, regardless of what terminology is used to reference it.

### B. Implant Based Weight Loss Assistance

Weight loss programs involving implanting a device in a patient are constantly under development. Examples of implanting into a patient's body to aid in weight loss include gastric bypass, implantable sensors that send electrical impulses to the stomach to stimulate feelings of fullness, a device for slowing the natural emptying of the stomach to prolong feelings of fullness, a device for preventing food from coming in contact with the digestive tract, a device that takes the shape of the small intestine to regulate feelings of fullness, and more.

### C. Naltrexone for Weight Loss

Patients may benefit from the use of naltrexone for weight loss.

### C.1 Naltrexone Lowers Insulin Levels

Using Naltrexone has helped to lower fasting insulin levels by up to 40%. One of the primary causes of weight gain in many patients, especially hypothyroid patients, is insulin resistance. Any therapy that leads to a decrease in insulin levels may aid significantly with weight loss. In addition to insulin resistance, female patients with high testosterone levels (i.e., high androgens) commonly fall on the PCOS spectrum and the increased androgens contribute to excessive coarse hair growth, depression, weight gain, and the like. Naltrexone modulates cellular resistance to insulin (i.e., decreased insulin resistance), and since insulin resistance plays a direct role in high testosterone levels in females-naltrexone may have multiple benefits in this particular patient group.

### C.2 Naltrexone Increases Growth Hormone

Some studies have shown that naltrexone may help to increase growth hormone levels. This is important because growth hormone helps to not only build and maintain lean muscle mass, but it also helps to increase fat burning; both of which can help with weight loss. The increase in weight with a decrease in growth hormone decreases is indicator that weight gain (and weight loss) is mediated by hormonal factors and not just simply related to calories consumed. Also, as insulin increases growth hormone decreases. Naltrexone helps to decrease insulin levels which may improve growth hormone levels.

### C. 3 Naltrexone Modulates Appetite

Chronic calorie restriction fails a majority of the time in sustaining weight loss in a patient. Chronic calorie restriction results in increased leptin levels and decreased free T3 levels and other hormone changes which lead to weight gain over time. It is also well known that chronic calorie restriction leads to a compensatory metabolic change which results in a damaged metabolism that may persist for years. Prescription weight loss medications target either appetite or metabolism and do not provide long term success.

Under normal physiologic influence a patient's body tries to match the amount of food consumed with a resting energy expenditure (i.e., the metabolism). In weight loss patients, there tends to be a significant mismatch between calories burned and appetite, and naltrexone may help to normalize the mismatch by controlling cravings in the patients.

### C. 4 Naltrexone as Anti-Inflammatory Agent

Naltrexone helps to reduce inflammation and can be used as a novel anti-inflammatory agent. Inflammation is an important factor when it comes to weight gain and weight loss resistance. Inflammation may lead to the following: decreased T4 to T3 conversion (leading to a state of thyroid resistance); increased insulin resistance (leading to weight gain); low testosterone and increased expression of aromatase leading to high estrogen levels; increased leptin levels (leading to weight gain); and increased appetite (leading to weight gain). By relieving inflammatory cytokines and markers of inflammation hormone levels become more regulated and balanced which may allow easier weight loss. The use of naltrexone in patients with chronic pain may allow them to come off of narcotics which improves metabolism and inflammation overall.

### C. 5 Naltrexone Improves Sleeping Patterns

Lack of sleep leads to inflammation and weight gain. Sleep apnea can lead to increased weight gain, but also weight gain can lead to sleep apnea. Naltrexone has helped improve sleeping patterns significantly in patients with sleep apnea. Naltrexone helps improve sleep in patients with complex regional pain syndrome and other chronic pain syndromes as well.

### C. 6 Naltrexone and Thyroid Function

Thyroid function is a very important consideration for patients who are struggling to lose weight and who also have hypothyroidism or Hashimoto's thyroiditis. Naltrexone can help to increase total T3 levels and improve T4 to T3 conversion.

### D. Naltrexone Implants

Naltrexone implants to date have been used for the treatment of opioid, alcohol, substance, or other addiction.

### E. Naltrexone Implant for Weight Loss

The present disclosure is directed to the use of a subcutaneous biodegradable medical implant comprising naltrexone (e.g., naltrexone HCL, naltrexone base) that, when implanted in a patient and optionally combined with behavioral and nutritional counseling/therapy, aids in weight loss in the patient. It will be appreciated that, in some embodiments, the subcutaneous biodegradable medical implant may comprise other excipients and/or non-active ingredients as part of the manufacturing process as well as a small amount of steroid to prevent inflammation. It will also be appreciated that the present disclosure is also directed to subcutaneous semi-biodegradable and non-biodegradable medical implants comprising naltrexone used for aiding in weight loss in a patient.

In embodiments of the present disclosure, an implant comprising naltrexone is inserted just beneath a surface of the skin in a lower abdominal area or other area of a patient. The implant comprising naltrexone may biodegrade into the blood stream, eliminating the requirement for removal of the implant, over a varying number of days or months depending on the metabolism of the patient. The implant comprising naltrexone provides a sustained release of naltrexone into the blood stream of the patient. The implant comprising naltrexone provides a gradually descending sustained level of release of naltrexone into the blood stream of the patient over the course of treatment. Such sustained release of naltrexone into the blood stream overcomes several drawbacks associated with oral-based medication administration weight loss systems.

Undesirable side effects associated with extreme weight loss usually are not life threatening, but they can be so unpleasant and disturbing that they discourage a lot of patients from continuing weight loss therapy. Although naltrexone doesn't relieve the undesirable side effects, it can make it easier to stick with a weight loss program.

The present implants comprising naltrexone eliminate the need for oral administration, which eliminates the need for a patient's liver to process the drug. Such a bypass is significantly beneficial for those patients with fatty liver disease and other conditions that would prohibit a patient from processing naltrexone in a healthy manner. An implant opens the door for patients who may not otherwise be candidates for naltrexone. Additionally, oral administration tends to require a higher dosage than is required when using an implant.

Further, in an oral-based medication administration weight loss system, noncompliance with the medication plan is a common issue. Reasons for non-compliance include a patient forgetting to take the medication at the scheduled time (e.g., forgetting to take the medication every day; forgetting to take the medication at the same time each day). Such noncompliance significantly reduces likelihood of long term success of a weight loss program.

F. Naltrexone Implant Paired with Behavioral Counseling/Therapy

Embodiments of the present disclosure pair the subcutaneous biodegradable medical implant comprising naltrexone with behavioral counseling/therapy provided by a certified or licensed professional. Such a pairing is necessary due to the short term releasing of naltrexone into the blood stream as a result of the subcutaneous biodegradable medical implant comprising naltrexone disintegrating over a varying number of months. It is unrealistic for a patient to continue to receive a replacement subcutaneous biodegradable medical implant comprising naltrexone every few months for life, necessitating an educational component if long term weight loss success is to be achieved.

Patients desiring the subcutaneous biodegradable medical implant comprising naltrexone must meet certain physical requirements in order for the implant to be safe and successful. Examples of such physical requirements include specific liver enzyme levels, and the patient should not be taking any opioids.

### Exemplary System for Implementing Embodiments of the Present Disclosure

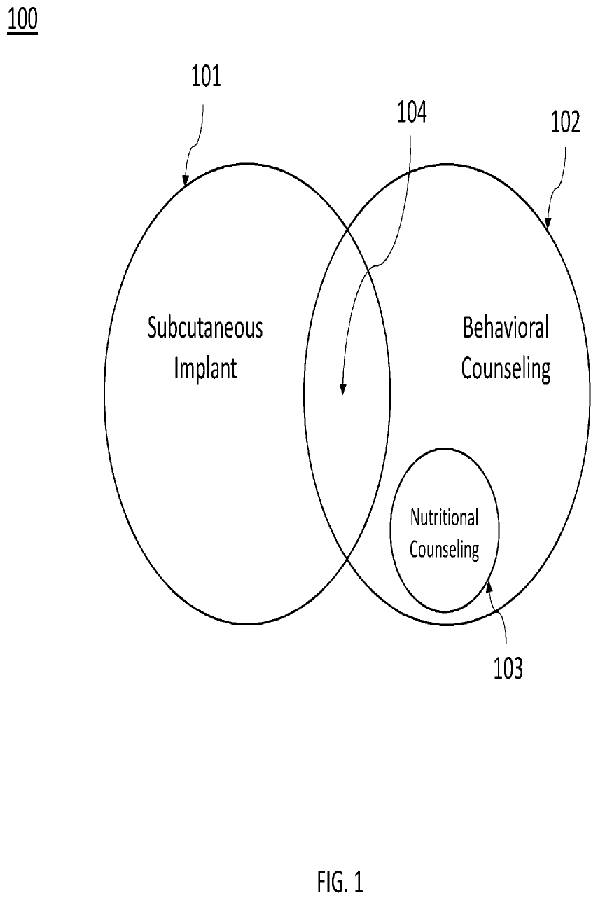

FIG. 1 is a diagram of an exemplary system for aiding weight loss in a patient according to embodiments of the present disclosure. In an exemplary system 100 for aiding weight loss in a patient, a subcutaneous biodegradable medical implant comprising naltrexone 101 is placed in a patient's abdomen. The implant 101 is considered to be most successful in aiding in long term weight loss when paired with behavioral counseling and/or therapy 102. In embodiments, behavioral counseling and/or therapy 102 comprises nutritional counseling and/or therapy 103. That is, the combination 104 of the implant 101 and behavioral counseling and/or therapy 102 is the preferred embodiment for success of the system disclosed herein.

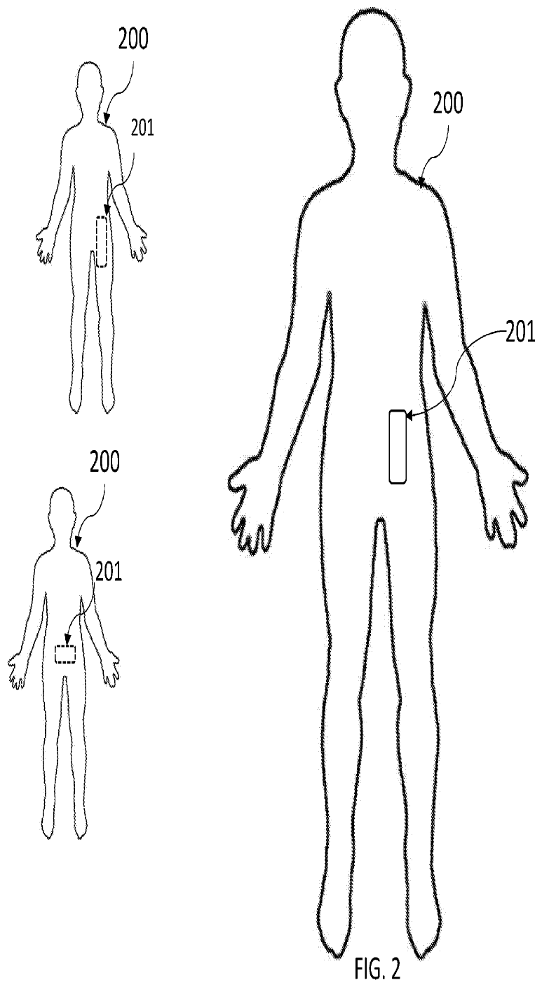

FIG. 2 is a diagram of an exemplary subcutaneous implant placed in a patient according to embodiments of the present disclosure. In embodiments of the present disclosure, a subcutaneous biodegradable medical implant comprising naltrexone 201 is placed into a patient 200. It will be appreciated that, while implant 201 is shown as having been placed into an abdominal area of patient 200, embodiments including placement of the implant into other areas of patient 200 are within the spirit of the present disclosure (e.g., lower abdominal area, hip area, as shown in FIG. 2). It will also be appreciated that implant 200 is not drawn to scale in FIG. 2 or in any of the figures herein.

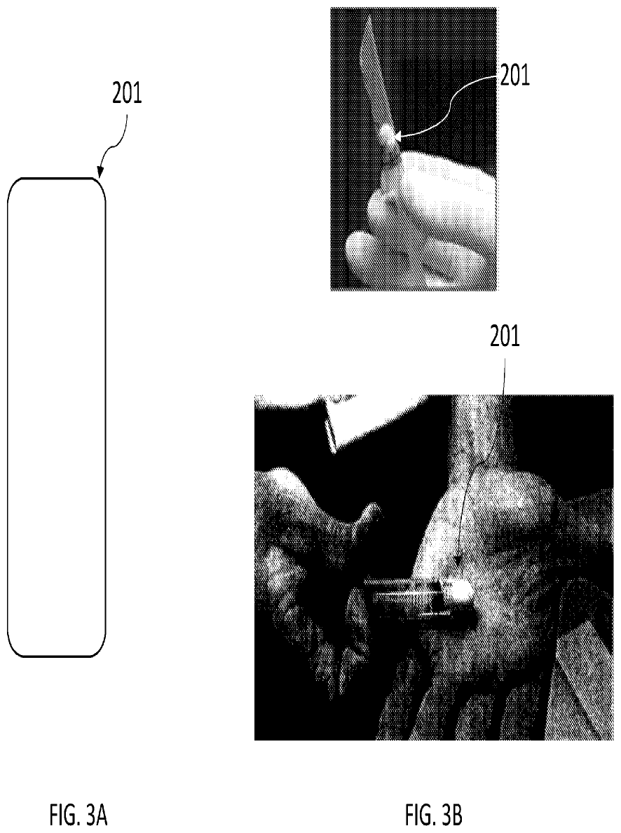

FIG. 3A illustrates an exemplary subcutaneous implant 201 for use with embodiments of the present disclosure. FIG. 3B illustrate photographs of exemplary subcutaneous implants for use with embodiments of the present disclosure. The subcutaneous implant 201 comprises naltrexone and is capable of releasing the naltrexone from the subcutaneous implant 201 following placement of the subcutaneous implant 201 in a patient. As explained above, the naltrexone aids in weight loss of the patient by blocking cravings, thereby resulting in reduced caloric intake.

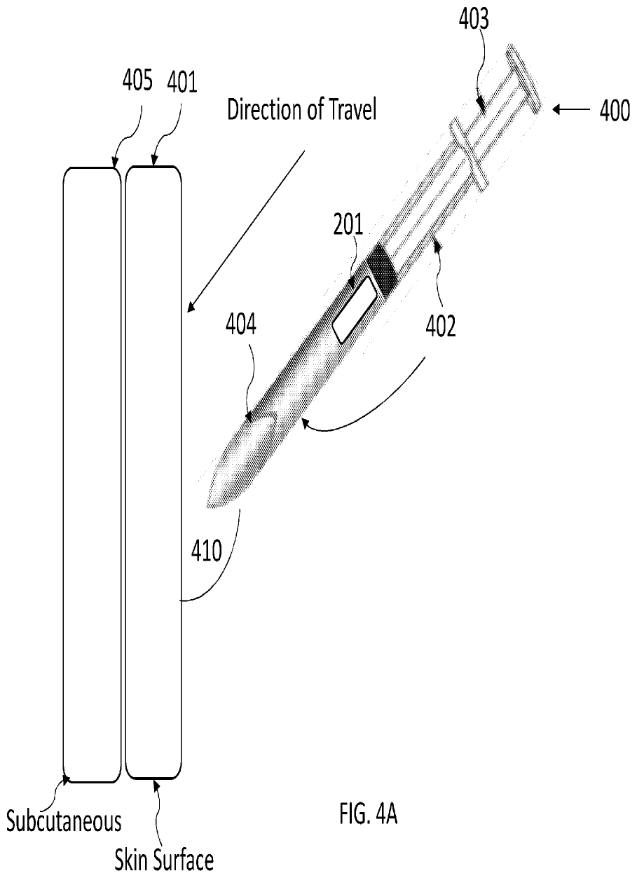

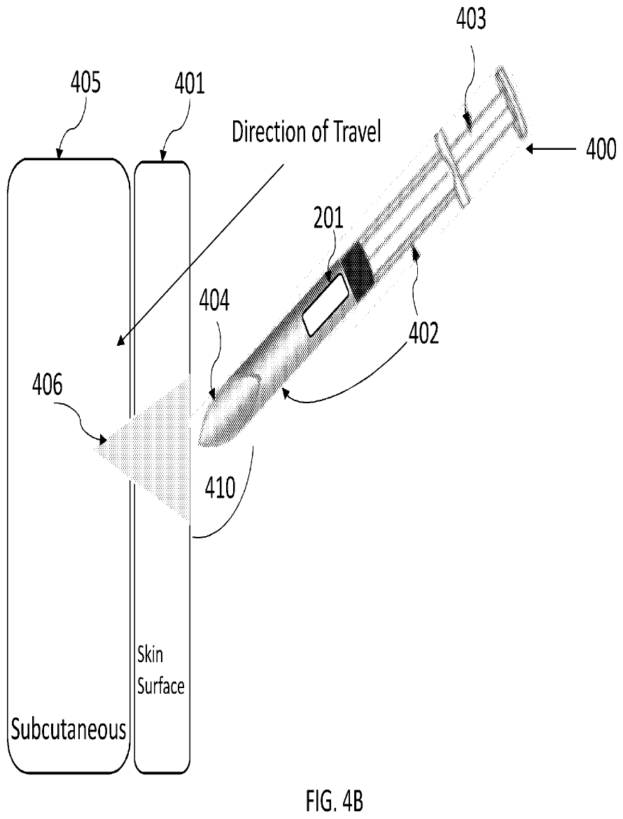

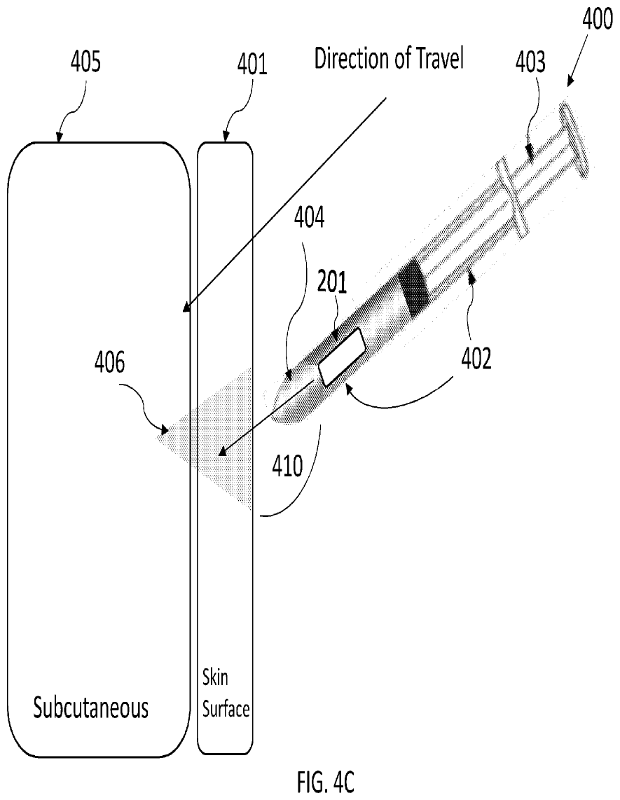

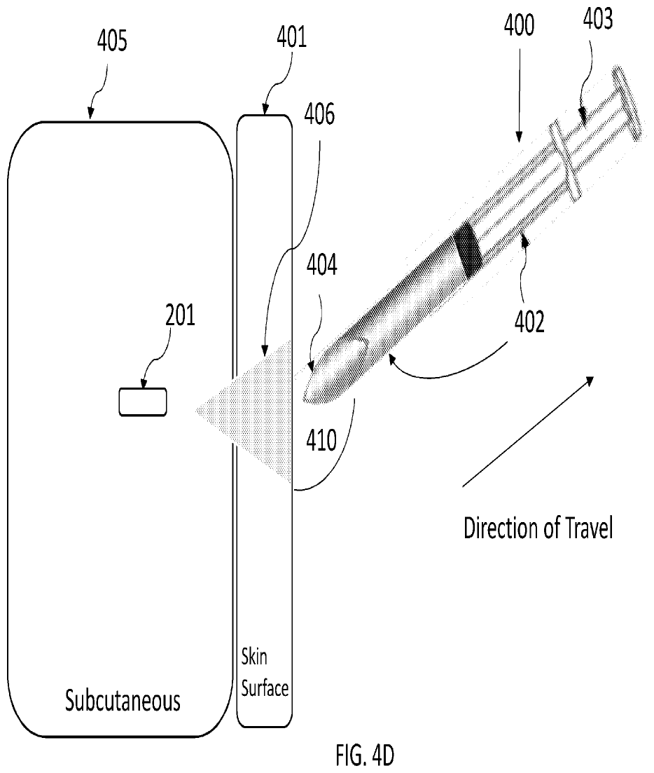

FIGS. 4A-4D illustrate an exemplary process for placing a subcutaneous implant in a patient according to embodiments of the present disclosure. FIGS. 4A-4D illustrate a series of drawings representing insertion of a subcutaneous biodegradable (or semi-or non-biodegradable) medical implant 201 in a patient, specifically below a skin surface 401 into a subcutaneous region 405. The subcutaneous biodegradable medical implant 201 is inserted using an insertion device 400 (e.g., an applicator or trocar) having a beveled end 404 after an incision 406 is made into the skin surface 401 and continuing into the subcutaneous region 405. The insertion device or applicator 400 has a barrel 402 for housing the implant 201 and a plunging end 403 for pressing the implant 201 through the beveled end 404 and into the subcutaneous region 405. The insertion device 400 can be removed once the implant 201 is placed into the subcutaneous region 405.

In some embodiments, the insertion device or applicator 400 comprises a trocar.

In some embodiments, the insertion device 400 enters the skin surface 401 at an angle 410. In some embodiments, angle 410 is 45 degrees.

In some embodiments, an exemplary process for placing a subcutaneous implant in a patient comprises insertion from a vial and not a beveled applicator. It will be appreciated that any method for insertion of the biodegradable subcutaneous medical implant chosen by a medical professional is within the scope of the present disclosure.

### Exemplary Patient Study

A randomized, single-dose pharmacokinetic (PK) study has been conducted of a naltrexone 800 mg subcutaneous implant in 6 overweight adults. By overweight, what is meant is each adult had a body mass index (BMI) at the outset of the study of 25 or higher. The objective of the study was to determine the pharmacokinetic and pharmacodynamics profiles of a single dose naltrexone implant 800 mg.

The group size was 6 human subjects aged 27 to <55; comprising 2 males and 4 females. The subjects were overweight ranging in weight from 180 to 289 pounds The subjects were located in the United States. The 800 mg naltrexone pellet(s) (long lasting naltrexone) were implanted under local anesthetic subcutaneously.

Subjects were seen for initial screening appointments with a physician (i.e., M.D.). At a first visit, each subject had a consultation and examination consisting of having their weight recorded, as well as history of eating disorders, habits, binge history, weight fluctuations and health history including all other addiction history. Each subject provided an initial blood sample for liver function tests.

Participation requirements included questionnaires and outcome measurements. The primary outcome measurements for this study is weight loss and reduced food cravings. Each subject was provided a randomized number for identification that is used on all records and questionnaires. Naltrexone blood levels were measured every 7 days from the date of implant placement. Telephonic weekly follow-ups were performed by subject sponsors to monitor implant site reaction (if any), foods cravings, and weight loss (self-reported). Data collection occurred as follows: week 1, 2, 3, 4.

At the time of this filing, three subjects have completed 4 weeks and two subjects have lost 6 and 8 lbs. The plasma concentrations for both the parent drug naltrexone and its metabolite 6-beta naltrexol are correlating well with the weight loss. The clinical signs of treatment are decreased appetite, nausea, vomiting, insomnia and headache. None of these effects are considered serious and the study is progressing satisfactorily.

Tables 1-7 below provide information regarding the patient study as well as data collected from the study.

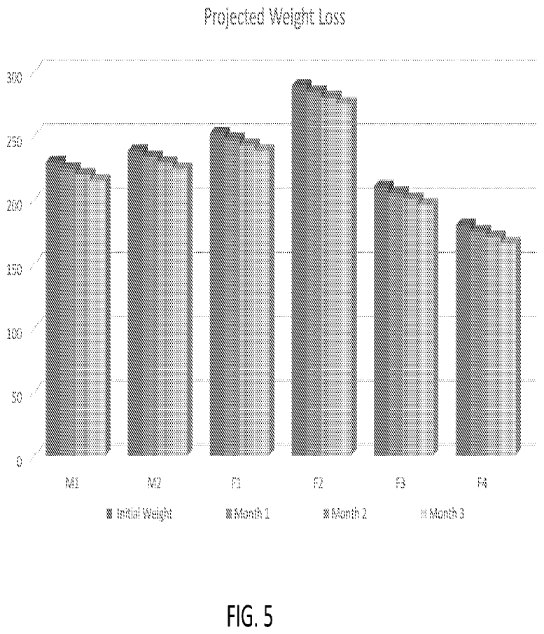

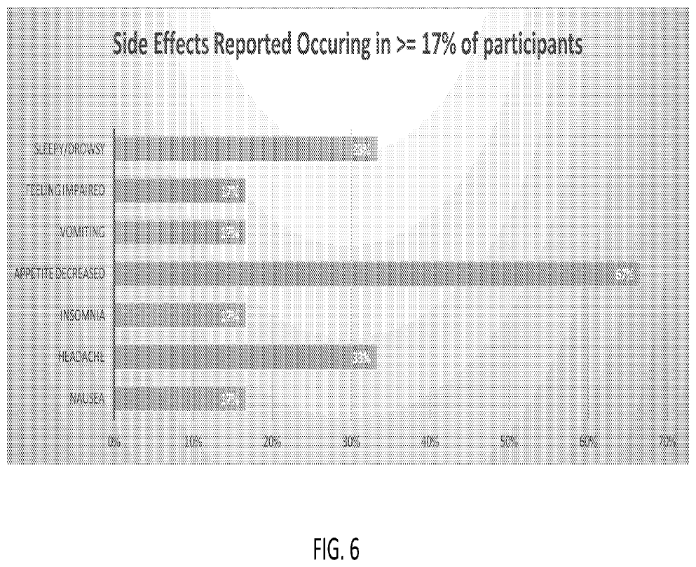

FIG. 5 illustrates projected weight loss for the subjects based on collected data after 4 weeks of the study. FIG. 6 illustrates data representing side effects reported by the subjects of the study.

The exemplary patient study results below indicate that only a single patient of the study experienced an actual reduction in BMI (see Table 7) of less than 2%; all others experienced at least a 2% reduction in BMI after measurements at 1 month (i.e., 4 weeks) of the study. That is, after the first month of the study, patients experienced a reduction in BMI of between 1.7% and 2.7%.

The exemplary patient study results serve as a basis for projecting additional weight loss over additional subsequent months of the study, where it can be observed that after 2 months (i.e., 8 weeks) of the study the patients are projected to experience a reduction in BMI of between 3% and 4.9%. Further, after 3 months (i.e., 12 weeks) the patients are projected to experience a reduction in BMI of between 4.7% and 7.6%. It will be appreciated that the data in Tables 6 and 7 for “Month 1” are from actual data measurements, and the data for “Month 2” and “Month 3” are projections based on the data measurements at “Month 1.”

### Exemplary Patient Study Results

TABLE 1
Patient Study Information

| | |
|---|---|
| Sponsor | BioCorRx |
| Study | Naltrexone and 6β-Naltrexol |
| Matrix | Noviplex Plasma Card, Dried Plasma |
| Range | 0.1-2000 ng/mL for Naltrexone and 6βNaltrexol |

TABLE 2
Naltrexone and 6β-Naltrexol Blood Level Test Results

| | | | | Naltrexone | 6β-Naltrexol |
|---|---|---|---|---|---|
| Specimen | Subject | Date | Time | (ng/mL) | (ng/mL) |
| 7004597 | 2598 | Aug. 29, 2018 | 10:00 | 12.3 | 16.7 |
| 7004678 | 2598 | Sep. 4, 2018 | 8:45 | 8.34 | 18.5 |
| 7004693 | 2598 | Sep. 11, 2018 | 10:20 | 5.30 | 14.9 |
| 7004731 | 2598 | Sep. 18, 2018 | 12:00 | 4.31 | 9.84 |
| 7004719 | 2598 | Sep. 24, 2018 | 9:00 | 2.51 | 6.26 |
| 7004736 | 6731 | Sep. 11, 2018 | — | 3.17 | 7.14 |
| 7004231 | 6731 | Sep. 24, 2018 | — | 3.59 | 6.66 |
| 7004599 | 7863 | Sep. 11, 2018 | — | 3.65 | 11.1 |
| 7004233 | 5391 | Sep. 10, 2018 | — | 4.80 | 8.79 |
| 7004687 | 5391 | Sep. 24, 2018 | — | 4.71 | 7.79 |
| 7004733 | 4189 | Sep. 20, 2018 | — | 2.40 | 5.33 |
| 7004238 | 4189 | Sep. 24, 2018 | — | 2.75 | 5.64 |
| 7004237 | 4521 | Sep. 4, 2018 | — | 858 | 22.4 |
| 7004594 | 4521 | Sep. 24, 2018 | — | 2.76 | 5.82 |

TABLE 3
Naltrexone Blood Level Test Results Analysis
Naltrexone

| | | | | | | | | |
|---|---|---|---|---|---|---|---|---|
| Standards | 1 | 2 | 3 | 4 | 5 | 6 | 7 | 8 |
| Nominal Conc (ng/mL) | 0.1 | 0.2 | 1 | 2 | 10 | 20 | 100 | 200 |
| Concentration (ng/mL) | 0.101 | 0.282* | 0.877 | 2.00 | 11.2 | 19.2 | 108 | 191 |
| % Accuracy | 101 | 141 | 87.7 | 100 | 112 | 95.8 | 108 | 95.3 |

| | | | |
|---|---|---|---|
| Quality Control | Low | Mid | High |
| Nominal Conc (ng/mL) | 0.5 | 5 | 50 |
| 1 | 0.486 | 4.65 | 48.1 |
| 2 | 0.504 | 4.69 | 50.8 |
| 3 | 0.490 | 5.22 | 54.7 |
| Ave | 0.493 | 4.85 | 51.2 |
| % Nominal | 98.7 | 97.0 | 102 |
| Std Dev | 0.009 | 0.319 | 3.30 |
| % RSD | 1.92 | 6.57 | 6.45 |

*excluded

TABLE 4
6β Naltrexol Blood Level Test Results Analysis
6β-Naltrexol

| | | | | | | | | |
|---|---|---|---|---|---|---|---|---|
| Standards | 1 | 2 | 3 | 4 | 5 | 6 | 7 | 8 |
| Nominal Conc (ng/mL) | 0.1 | 0.2 | 1 | 2 | 10 | 20 | 100 | 200 |
| Concentration (ng/mL) | 0.107 | 0.174 | 0.917 | 2.25 | 10.7 | 18.6 | 103 | 197 |
| % Accuracy | 107 | 87.2 | 9.7 | 113 | 107 | 93.1 | 03 | 98.4 |

| | | | |
|---|---|---|---|
| Quality Control | Low | Mid | High |
| Nominal Conc (ng/mL) | 0.5 | 5 | 50 |
| 1 | 0.47 | 4.59 | 49.0 |
| 2 | 0.427 | 4.76 | 53.3 |
| 3 | 0.453 | 5.06 | 54.6 |
| Ave | 0.450 | 4.81 | 52.3 |
| % Nominal | 90.0 | 96.1 | 105 |
| Std Dev | 0.022 | 0.239 | 2.93 |
| % RSD | 4.81 | 4.98 | 5.61 |

*excluded

TABLE 5
Patient Study Weight Loss Data

| | | | | | Projected | |
|---|---|---|---|---|---|---|
| | | Initial | | | Monthly | Projected |
| Participant | | Weight | at 2 weeks | at 4 weeks | Weight Loss | Monthly |
| Number | Gender | lbs | lbs | lbs | lbs | Weight Loss |
| 7863 | male | 229 | 224 | 223 | 6 | |
| 2598 | male | 238 | 238 | 238 | 0 | |
| 4521 | female | 252 | 246 | 244 | 8 | |
| 4189 | female | 289 | 286 | Oct. 11, 2018 | 3 | 6 |
| 5391 | female | 210 | 206 | Oct. 5, 2018 | 4 | 8 |
| 6731 | female | 180 | 180 | Oct. 4, 2018 | 0 | 0 |
| | | | | Average Monthly | 4.666666667 | 4.666666667 |
| | | | | Weight loss: | | |

TABLE 6
Patient Study Body Mass Index Results Data
Projected Body Mass Index (BMI)

| Participant | Initial BMI | Month 1 | Month 2 | Month 3 |
|---|---|---|---|---|
| 7863 | 34.8 | 34.1 | 33.5 | 32.7 |
| 2598 | 34.1 | 33.4 | 32.9 | 32.1 |
| 4521 | 35.1 | 34.4 | 33.9 | 33.2 |
| 4189 | 46.6 | 45.8 | 45.2 | 44.4 |
| 5391 | 33.9 | 33.1 | 32.4 | 31.6 |
| 6731 | 32.9 | 32 | 31.3 | 30.4 |

TABLE 7
Patient Study Body Mass Index Percentage Decrease Results Data
Projected Decrease - Body Mass Index (BMI)

| Participant | Initial BMI | Month 1 | Month 2 | Month 3 |
|---|---|---|---|---|
| 7863 | 34.8 | −2.0% | −3.7% | −6.0% |
| 2598 | 34.1 | −2.1% | −3.5% | −5.9% |
| 4521 | 35.1 | −2.0% | −3.4% | −5.4% |
| 4189 | 46.6 | −1.7% | −3.0% | −4.7% |
| 5391 | 33.9 | −2.4% | −4.4% | −6.8% |
| 6731 | 32.9 | −2.7% | −4.9% | −7.6% |

TABLE 7
Patient Study Weight Loss Data

| | Initial | Weight at | weight at | Weight at | weight at | weight at |
|---|---|---|---|---|---|---|
| Participants | Weight | 2 weeks | 4 weeks | 8 weeks | 12 weeks | 6 months |
| 1 | 147.6 | 0 | 0 | 0 | 0 | |
| 2 | 340 | 334 | 0 | 0 | 0 | |
| 3 | 248 | 244 | 239.7 | 222 | 228 | 225.5 |
| 4 | 349.9 | 337.81 | 327.9 | 320.1 | 312.2 | 303.3 |
| 5 | 222.2 | 0 | 0 | 0 | 0 | |
| 6 | 283.6 | 279.9 | 275\| | 275 | 270.1 | 270 |
| 7 | 299.4 | 0 | 0 | 0 | 0 | |
| 8 | 259.2 | 257.7 | 249 | 0 | 0 | 294 |
| 9 | 177.6 | 173.3 | 171 | 162.2 | 160.1 | 160 |
| 10 | 170.4 | 166.8 | 165.1 | 163 | 160.2 | 160.1 |
| 11 | 245.1 | 237.6 | 228.4 | 220.1 | 215 | 215 |
| 12 | 277.6 | 275 | 268.1 | 260.6 | 255.1 | 257 |
| 13 | 199.4 | 183.9 | 178.1 | 175 | 161.1 | 159.5 |
| 14 | 231.9 | 227.2 | 222 | 219.1 | 217.8 | 217 |
| 15 | 298.9 | 289.1 | 278.8 | 275.9 | 275 | 278.1 |
| 16 | 259.2 | 249.2 | 249.2 | 244 | 241.1 | 249 |
| 17 | 256.9 | 245.7 | 245.7 | 230 | 234 | 245 |
| 18 | 225 | 0 | 0 | 0 | 0 | |

### Conclusion

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A subcutaneous biodegradable medical implant for aiding weight loss in a patient, the subcutaneous biodegradable medical implant comprising naltrexone (C₂₀H₂₃NO₄), wherein subcutaneous biodegradable medical implant is capable of releasing a dosage amount of the naltrexone from the subcutaneous biodegradable medical implant following placement of the subcutaneous biodegradable medical implant in a patient, and wherein the naltrexone aids in weight loss of the patient.

2. The subcutaneous biodegradable medical implant of claim 1, configured to release a dosage amount of naltrexone in an amount in a range of 200 mg to 4 grams into a bloodstream of the patient.

3. The subcutaneous biodegradable medical implant of claim 1, configured to release a dosage amount of naltrexone in an amount of one of 1 gram, 1.1 grams or 2.2 grams into a bloodstream of the patient.

4. The subcutaneous biodegradable medical implant of claim 1, wherein the subcutaneous biodegradable medical implant biodegrades after a period of about 30 days in the patient.

5. The subcutaneous biodegradable medical implant of claim 1, wherein the subcutaneous biodegradable medical implant is placed or injected below a skin surface of the patient.

6. The subcutaneous biodegradable medical implant of claim 5, wherein the subcutaneous biodegradable medical implant is placed or injected above a muscle fascia of the patient.

7. The subcutaneous biodegradable medical implant of claim 1, wherein the patient receives behavioral counseling and/or therapy from a licensed professional prior to or from a placement time in the patient until one of a biodegradation time or after a biodegradation time.

8. The subcutaneous biodegradable medical implant of claim 1, wherein the patient experiences a reduction in body mass index (BMI) of one of at least 2% after about four weeks from a placement time in the patient, at least 3% after about 8 weeks from a placement time in the patient, or at least 4.5% after about 12 weeks from a placement time in the patient.

9. The subcutaneous biodegradable medical implant of claim 7, wherein the behavioral counseling and/or therapy comprises nutritional counseling and/or therapy.

10. The subcutaneous biodegradable medical implant of claim 1, wherein the subcutaneous medical implant comprises a single implant unit configured to release a dosage amount of the naltrexone into a bloodstream of the patient.

11. The subcutaneous biodegradable medical implant of claim 1, wherein the subcutaneous medical implant comprises a plurality of implant units configured to release a dosage amount of the naltrexone into a bloodstream of the patient.

12. A system for aiding weight loss in a patient, the system comprising:
a subcutaneous biodegradable medical implant placed in the patient, the subcutaneous biodegradable medical implant comprising naltrexone (C₂₀H₂₃NO₄) and capable of releasing a dosage amount of the naltrexone from the subcutaneous biodegradable medical implant following the placement of the subcutaneous biodegradable medical implant in the patient; and
a program comprising behavioral counseling and/or therapy delivered by a licensed professional to the patient.

13. The system of claim 12, wherein the subcutaneous biodegradable medical implant comprising naltrexone releases a dosage amount of naltrexone in an amount in a range of 200 mg to 4 grams into a bloodstream of the patient. 14 The system of claim 12, wherein subcutaneous biodegradable medical implant comprising naltrexone releases a dosage amount of naltrexone in an amount of one of 1 gram, 1.1 grams or 2.2 grams into a bloodstream of the patient.

15. The system of claim 12, wherein the subcutaneous biodegradable medical implant biodegrades after a period of about 30 days in the patient.

16. The system of claim 12, wherein the subcutaneous biodegradable medical implant is placed below a skin surface of the patient.

17. The system of claim 16, wherein the subcutaneous biodegradable medical implant is placed below a skin surface of and above a muscle fascia of the patient.

18. The system of claim 12, wherein the licensed professional delivers behavioral counseling and/or therapy to the patient prior to or from a placement time in the patient until one of a biodegradation time or after a biodegradation time.

19. The system of claim 12, wherein the patient experiences a reduction in body mass index (BMI) of one of at least 2% after about four weeks from a placement time in the patient, at least 3% after about 8 weeks from a placement time in the patient, or at least 4.5% after about 12 weeks from a placement time in the patient.

20. The system of claim 12, wherein the behavioral counseling and/or therapy comprises nutritional counseling and/or therapy.
